(19) United States
(12) Patent Application Publication
(10) Pub. No.: US 2010/0081668 A1
(43) Pub. Date: Apr. 1, 2010

(54) POLYMORPHS OF 5--6-CHLORO-1,3-DIHYDRO-2H-INDOL-2-ONE HYDROBROMIDE AND PROCESSES FOR PREPARATION THEREOF

(76) Inventors: József Neu, Budapest (HU); Adám Demeter, Budapest (HU); Zoltán Varga, Budapest (HU); Balázs Havasi, Budapest (HU); Sándor Garadnay, Esztergom (HU); Béla Hegedüs, Budapest (HU)

Correspondence Address: HAHN & VOIGHT PLLC, 1012 14TH STREET, NW, SUITE 620, WASHINGTON, DC, 20005, US

(21) Appl. No.: 12/516,094

(22) PCT Filed: Nov. 23, 2007
(86) PCT No.: PCT/HU07/00112

§ 371 Date: May 22, 2009

(30) Foreign Application Priority Data
Nov. 24, 2006 (HU) P0600868

(51) Int. Cl. A61K 31/496; C07D 417/14; A61P 25/00

(52) U.S. Cl. 514/254.04; 544/368

(57) ABSTRACT

The present invention provides pharmaceutically applicable compounds and polymorphs belonging to the ziprasidone hydrobromide compound group with antipsychotic effect. The present invention provides hydrobromide polymorphs of 5-{-2-[4-(1,2-benzisothiazol-3-yl)-1-piperazinyl]-ethyl}-6-chloro-1,3-dihydro-2H-indol-2-one, ziprasidone of Formula (I) having neuroleptic activity.

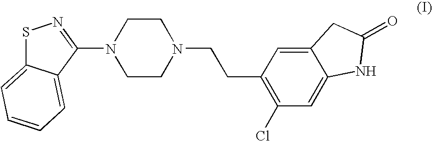

### FIELD OF THE INVENTION

The field of the invention relates to pharmaceutically applicable compounds and polymorphs being classed among the ziprasidone hydrobromide compound group known to have strong antipsychotic effect. Ziprasidone is an antipsychotic agent that is chemically 5-{2-[4-(1,2-benzisothiazol-3-yl)-1-piperazinyl]-ethyl}-6-chloro-1,3-dihydro-2H-indol-2-one of Formula I.

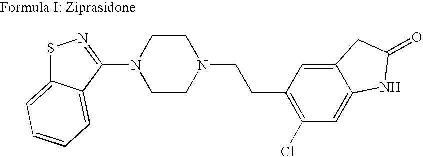

The field of the invention relates to new ziprasidone-hydrobromide compounds: ziprasidone-hydrobromide monohydrate, crystalline and amorphous ziprasidone-hydrobromide, ziprasidone-hydrobromide hemihydrate, ziprasidone-sesquihydrobromide hemiformiate.

The field of the invention also relates to five ziprasidone-hydrobromide polymorph forms pertaining to the ziprasidone hydrobromide group, Ziprasidone-hydrobromide Form I-V, and preparation processes thereof:
The field of the invention relates to Ziprasidone-hydrobromide Form I which is a crystalline modification of ziprasidone-hydrobromide monohydrate of Formula II.
The field of the invention also relates to Ziprasidone-hydrobromide Form II which is a crystalline modification of ziprasidone-hydrobromide anhydrate of Formula III.
The field of the invention also relates to Ziprasidone-hydrobromide Form III which is a crystalline modification of ziprasidone-hydrobromide hemihydrate of Formula IV.
The field of the invention also relates to Ziprasidone-hydrobromide Form IV which is a crystalline modification of ziprasidone-sesquihydrobromide hemiformiate of Formula V.
The field of the invention also relates to Ziprasidone-hydrobromide Form V which is amorphous modification of ziprasidone-hydrobromide of Formula III.

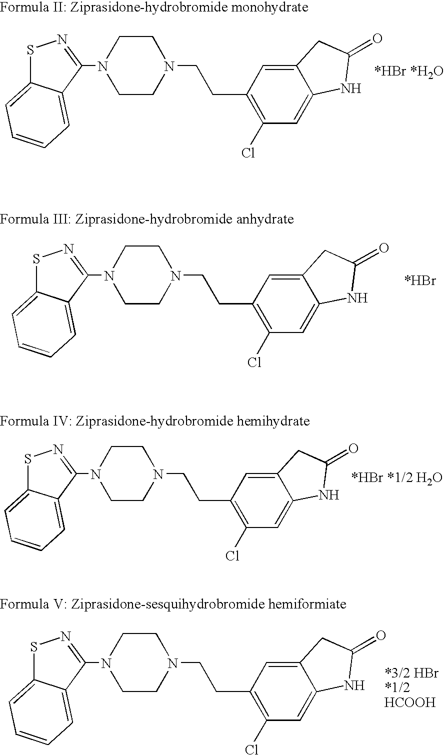

The field of the invention also relates to some economical preparation methods that are suitable for industrial production of high purity ziprasidone hydrobromide modifications. In a generally applicable method ziprasidone base is dissolved in formic acid, aqueous or non-aqueous hydrogen bromide is added, and the product is precipitated with the aid of an antisolvent. The circumstances of this general process determine which Form is prepared.

### BACKGROUND OF THE INVENTION

Ziprasidone, 5-{2-[4-(1,2-benzisothiazol-3-yl)-1-piperazinyl]-ethyl}-6-chloro-1,3-dihydro-2H-indol-2-one of Formula I and pharmaceutically acceptable salts thereof are disclosed in U.S. Pat. No. 4,831,031 (European equivalent: EP 0 281 309) and are known as neuroleptic active ingredients. Furthermore, it is known from U.S. Pat. No. 6,245,766 that these actives have excellent efficacy for the treatment of psychiatric states and disorders arising from demencia, among others Alzheimer-type demencia, and bipolar disorders. There are no data in the literature referring to that ziprasidone hydrobromide really has been prepared. Although, in the PCT Publication No. WO 2006/034965 there is an indirect remark on the existence of—among others—hydrobromide addition salt, but there are not data on its preparation, or physical, chemical characteristics. According to this description the addition salts can be discomposed by hydrogen chloride, hydrogen bromide, methanesulphonic acid, preferably by hydrogen chloride, and in the latter case especially pure ziprasidone hydrochloride can be obtained. In the description there are examples for ziprasidone maleate, ziprasidone acetate, and ziprasidone hydrochloride anhydrate, however there is no example on the ziprasidone hydrobromide salt group.

As ziprasidone hydrobromide preparation methods have not been published in the literature, the ziprasidone hydrochloride preparation methods are taken as the technical anteriority as follows:

According to Example 16 of U.S. Pat. No. 4,831,031 ziprasidone hydrochloride is obtained if 5-(2-chloroethyl)-6-chloro-1,3-dihydro-2H-indol-2-one is reacted with 3-piperazinyl-1,2-benzisothiazol hydrochloride in the presence of sodium-carbonate and sodium-iodide in methyl isobutyl ketone boiling the mixture for 40 hours. Then the reaction mixture is filtered, evaporated, and the residue is purified with chromatography. The evaporated residue of chromatography is dissolved in dichloromethane, and after acidification by hydrochloric acidic diethyl ether, the precipitated crystals are filtered out, washed with ether and acetone.

The obtained product is declared as 5-{2-[4-(1,2-benzisothiazol-3-yl)-1-piperazinyl]-ethyl}-6-chloro-1,3-dihydro-2H-indol-2-one hydrochloride hemi hydrate (ziprasidone hydrochloride hemihydrate).

This method is unusable for industrial production, however according to the procedure of European Patent No. EP 584 903 ziprasidone hydrochloride can be prepared at a high yield (80%) even in an industrial scale. In this procedure also the same components: 5-(2-chloroethyl)-6-chloro-1,3-dihydro-2H-indol-2-one and the hydrochloride salt of 3-piperazinyl-1,2-benzisothiazol are reacted with each other in the presence of sodium-carbonate, but in this case the solvent is simply water. Here the isolation is followed by a complicated clearing step.

European Patent No. EP 586 191 reveals a method according to which ziprasidone hydrochloride monohydrate is obtained in a reaction of the clean ziprasidone base with diluted aqueous hydrochloric acid solution.

The PCT Publication No. WO 2005/061493 reveals a preparation method of ziprasidone hydrochloride anhydrate. However, having reproduced this and similar other methods—for preparation ziprasidone hydrochloride anhydrate that would be stable in normal air conditions—only the unsuitability for industrial-scale application has been proved. Ziprasidone hydro-chloride anhydrate samples adsorbed water rapidly from air even if prepared in rigorously anhydrous circumstances.

These observations are in accordance with data of EP 586 191 (equivalent U.S. Pat. No. 5,338,846): that ziprasidone hydrochloride anhydrate (water content: 0.19%) can be prepared from ziprasidone hydrochloride monohydrate (water content: 3.9%) with drying for 28.5 hours (instead of 7 hours) at a temperature of 40-50° C. This application describes that ziprasidone hydrochloride anhydrate binds water depending on the relative humidity of air. For example at 31% relative humidity the water content of the product increased to 2.55% after 4 hours.

As in the practice only the monohydrate salt of the ziprasidone hydrochloride is relatively stable, and even the monohydrate can loose water, during the drying procedure generally a mixture of ziprasidone hydrochloride monohydrate and anhydrate is resulted. It is known that such a mixture does not provide an optimal base for a validated industrial production.

The occurrence of different crystal forms possesses different solid state properties, can have various stability, mechanical, physical properties like melting point, spectroscopic behaviours, their crystal habits and thermodynamic properties are different. These properties influence the applicability of the active ingredients for formulation purposes. Another important solid state feature is the solubility entailing therapeutic consequences. The discovery of new polymorphic forms of a pharmaceutically useful compound provides a new opportunity to improve the performance characteristics of a pharmaceutical product. It enlarges the repertoire of active materials for the formulation purposes. There is a need in the art for additional polymorphic forms of ziprasidone salts.

### SUMMARY OF THE INVENTION

The present invention provides pharmaceutically applicable compounds and polymorphs belonging to the ziprasidone hydrobromide compound group with antipsychotic effect. The present invention provides hydrobromide polymorphs of 5-{2-[4-(1,2-benzisothiazol-3-yl) -1-piperazinyl]-ethyl}-6-chloro-1,3-dihydro-2H-indol-2-one, ziprasidone of Formula I having neuroleptic activity.

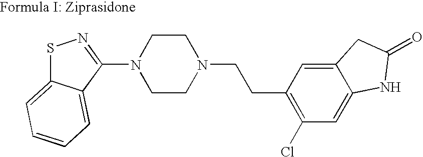

The invention discloses new ziprasidone hydrobromide compounds; ziprasidone hydrobromide monohydrate, crystalline and amorphous ziprasidone hydrobromide anhydrate, ziprasidone hydrobromide hemihydrate and ziprasidone sesquihydrobromide hemiformiate. The invention discloses the new polymorph modifications being classed among the ziprasidone hydrobromide compound group (Ziprasidone-hydrobromide Form I-V) and preparation methods thereof.

The new polymorphs show good stability, provide advantages in formulation, and with increasing diversity and choice range provide new possibilities to fulfill the demands of formulation and biological utilization.

### DETAILED DESCRIPTION OF THE INVENTION

Regarding the foregoing problems, the present invention has proceeded with extensive research.

During our experimental work we prepared different ziprasidone hydrobromide compounds and forms. It was found surprisingly that the stability of ziprasidone hydrobromide salt forms is regularly better than that of the similar ziprasidone hydrochloride forms. In suitable circumstances both the anhydrate and the monohydrate of ziprasidone hydrobromide can be prepared in stable forms, at normal humidity these products can easily be handled, meanwhile their water and active ingredient contents remain constant. The hemihydrate of ziprasidone hydrobromide can also be prepared reproducibly.

According to thermogravimetric (TG) and differential scanning calorimetric (DSC) investigations, during heating Ziprasidone-hydrobromide Form I (a ziprasidone hydrobromide monohydrate) loses water between 40-60° C., however after the heat-effect it readily takes back the water, and transforms back to monohydrate of the original crystal structure. If a Ziprasidone-hydrobromide Form I (monohydrate) sample or a Ziprasidone-hydrobromide Form II (anhydrate) sample is taken into a 100% relative humidity area at room temperature, their water content does not change significantly. The Ziprasidone-hydrobromide Form III (hemihydrate) sample was also stable in these circumstances.

Having revealed the advantageous characters of these modifications, we discovered further modifications of the ziprasidone-hydrobromide family. The new polymorph forms can provide new possibilities to enhance the performance characteristics of a pharmaceutical product. The up-to-date formulation technology requires the availability of the new polymorphs of the active ingredients. During our experiments we produced reproducibly Ziprasidone-hydrobromide Form IV (a ziprasidone sesquihydrobromide hemiformiate) and Ziprasidone-hydrobromide Form V (an amorphous modification of ziprasidone hydrobromide).

Our experiments proved that both ziprasidone hydrobromide monohydrate and ziprasidone hydrobromide anhydrate can be produced equally starting from homogeneous or heterogeneous reaction mixtures. According to a pharmaceutically advantageous solution ziprasidone base solution is reacted by aqueous or acetic acidic hydrogen bromide in such circumstances that promote the precipitation of the solid form. For the preparation of these polymorphs and for the dissolution of the ziprasidone base, formic acid is especially appropriate, however, the acetic acid (and the solutions of acetic acid in methanol, ethanol, tetrahydrofuran or ethyl acetate) can be utilized favorably, as well. For heterogeneous phase production a suspension can be formed using simple alcohols, advantageously with methanol or ethanol, with tetrahydrofuran or water, or other solvents containing tetrahydrofuran or water, more advantageously water.

The hydrogen bromide solution may be diluted favorably with water, acetic acid, alcohols, ethyl acetate, tetrahydrofuran, methyl isobuthyl keton.

Ziprasidone-hydrobromide Form I (ziprasidone hydrobromide monohydrate) can be prepared advantageously if a solution of ziprasidone base in a solvent of formic acid, acetic acid or including thereof is reacted with aqueous hydrogen bromide solution. The reaction is carried out advantageously at a room temperature or with a relatively short boiling, preferably for 0.5-3 hours. Then the mixture is cooled back to room temperature, after crystallisation the solid is filtered out, and dried. This product can be prepared from a suspension of ziprasidone base, as well, if a suspension, advantageously an alcoholic aqueous suspension of ziprasidone base is boiled for 0.5-3 hours. Then the mixture is cooled back to room temperature, the solid is filtered out, and dried. The product can also be produced in a similar manner if instead of ziprasidone base another ziprasidone hydrobromide salt is used, but in this case the hydrogen bromide is left out.

Ziprasidone-hydrobromide Form II (generally ziprasidone hydrobromide anhydrate) can be prepared advantageously, according to one aspect of the present invention, if an anhydrous solution, advantageously anhydrous formic acidic solution, of ziprasidone base is reacted with anhydrous hydrogen bromide solution, advantageously in anhydrous methanolic-glacial acetic acidic solution. The reaction is carried out advantageously at room temperature. After crystallization the solid is filtered out, and dried. According to another aspect of the present invention ziprasidone hydrobromide anhydrate can be prepared starting from an aqueous media if the water is removed from the reaction mixture in a long-lasting, advantageously in 8-20 hour's boiling. In these cases the ziprasidone base advantageously can be dissolved in formic or acetic acid, or in a solution including formic or acetic acid with methanol, ethanol, tetrahydrofuran or ethyl acetate. The reaction can be carried out in heterogeneous phases, as well. For this purpose suitable suspensions can be made with simple alcohols, advantageously with methanol or ethanol, or including thereof with other solvents, advantageously with methanol and tetrahydrofuran. The product can also be produced in a similar manner if instead of ziprasidone base another ziprasidone hydrobromide salt is used, but in this case the hydrogen bromide is left out. Ziprasidone-hydrobromide Form II can be especially advantageously produced from Ziprasidone-hydrobromide Form IV (generally ziprasidone sesquihydrobromide hemiformiate) with a short heating at a temperature between 180-200° C.

Ziprasidone-hydrobromide Form III (generally ziprasidone hydrobromide hemihydrate) can be prepared advantageously, according to one aspect of the present invention, if an anhydrous solution, advantageously anhydrous formic acidic solution, of ziprasidone base is reacted with anhydrous hydrogen bromide solution, advantageously in anhydrous methanolic-glacial acetic acidic solution. According to another aspect of the present invention ziprasidone hydrobromide hemihydrate can from Ziprasidone-hydrobromide Form I if the formic acidic solution of the latter in a very short time, advantageously in 1 min. is added to water of 5-10° C. temperature. Then the solid is filtered out immediately, and dried.

Ziprasidone-hydrobromide Form IV (generally ziprasidone sesquihydrobromide hemiformiate) can be prepared advantageously, according to one aspect of the present invention, if an aqueous hydrogen bromide solution containing other solvents, advantageously methyl isobutyl ketone, ethyl acetate or tetrahydrofuran is added in a relatively short time, advantageously in 15 min. into a formic acidic solution of ziprasidone base. Then the solid is filtered out, and dried.

Ziprasidone-hydrobromide Form V (generally amorphous ziprasidone hydrobromide) can be prepared advantageously, according to one aspect of the present invention, if a hydrogen bromide solution in glacial acetic acid containing methyl isobutyl ketone is added in very short time, advantageously in 1 min. into a formic acidic solution of ziprasidone base at 65-70° C., followed by a 16 hour's after-stirring, then the solid is filtered out, and dried.

In similar manners in different solvents other solvates and amorphous forms of ziprasidone hydrobromide can also be prepared.

From the compounds and polymorphs described above orally, rectally or parentally usable pharmaceutical forms, as tablets, capsules, aqueous and oily suspension or dispergable powder forms can be made with generally used non-toxic, pharmaceutically suitable diluting, carrier, binding, disperging or other auxiliary materials.

Recently polymorphism has become one of the most important fields of the pharmaceutical industry since it concerns almost all characteristics of the solid active ingredient, sometimes in a dramatic extent. For discovery, identification, and differentiation of the new ziprasidone hydrobromide polymorphs a lot of solid analytical and other instrumental investigation methods were used together in a complex way.

In our solid analytical methods the following instrumental circumstances were applied.

FT-IR Spectrophotometry Parameters:

| Instrument: | Thermo-Nicolet 6700 |
|---|---|
| Phase (solvent): | KBr |
| Resolution: | 4 cm⁻¹ |
| Scan number: | 100 |

The baselines of the spectra were normalized to absorbance of 1.0. Regarding the resolution of 4 cm⁻¹, the variance of the wavenumber values is not more than ±4 cm⁻¹.

Powder X-ray Diffraction Parameters:

| Instrument: | PANanalytical X'Pert PRO |
|---|---|
| Radiation: | CuK_{α} |
| Accelerating voltage: | 40 kV |
| Anode current: | 40 mA |
| Goniometer: | PW3050/60 |
| Recording speed: | 0.208°2θ/s |
| Sample holder: | Spinner PW3064 |
| Revolving speed: | 1 s⁻¹ |
| Variance of 2θ: | ±0.2° |

Thermogravimetry (TG) Parameters:

| | |
|---|---|
| Instrument: | TA Instruments TGA Q50 |
| Heating rate: | 10° C./min |
| Sample size: | ~10 mg |
| Atmosphere: | 60 ml/min Na |

Differential Scanning Calorimetry (DSC) Parameters:

| | |
|---|---|
| Instrument: | TA Instruments DSC Q10 |
| Heating rate: | 10° C./min |
| Sample size: | ~1.5-2.5 mg |
| Type of skillet: | opened |
| Atmosphere: | 50 ml/min N₂ |

### LIST OF FIGURES

FIG. 1: FT_IR spectrum of Ziprasidone-hydrobromide Form I

FIG. 2: FT_IR spectrum of Ziprasidone-hydrobromide Form II

FIG. 3: FT_IR spectrum of Ziprasidone-hydrobromide Form III

FIG. 4: FT_IR spectrum of Ziprasidone-hydrobromide Form IV

FIG. 5: FT_IR spectrum of Ziprasidone-hydrobromide Form V

FIG. 6: Powder X-ray diffraction diagram of Ziprasidone-hydrobromide Form I

FIG. 7: Powder X-ray diffraction diagram of Ziprasidone-hydrobromide Form II

FIG. 8: Powder X-ray diffraction diagram of Ziprasidone-hydrobromide Form III

FIG. 9: Powder X-ray diffraction diagram of Ziprasidone-hydrobromide Form IV

FIG. 10: Powder X-ray diffraction diagram of Ziprasidone-hydrobromide Form V

### EXAMPLES

The present invention is illustrated by the following examples without limiting the scope.

The water contents were determined with Karl Fischer titrimetric and/or thermogravimetric method. The HBr and formic acid contents were determined by titrimetry, and with the aid of a ¹³C-NMR method, respectively.

### Example 1

### Preparation of Ziprasidone-hydrobromide Form I

12,0 g 5-{2-[4-(1,2-benzisothiazol-3-yl) -1-piperazinyl]-ethyl}-6-chloro-1,3-dihydro-2H-indol-2-one (ziprasidone base) was dissolved in 48.0 ml formic acid at room temperature. The homogeneous solution was stirred with 0.6 g charcoal and 0.6 g silica gel 60 (particle size 0.040-0.063 mm) for 30 min, then it was filtered. The clear filtered solution was added into a mixture of 6.0 ml aqueous 48% (w/v) hydrogen bromide solution and 100 ml distilled water at 25-30° C. temperature, followed by an hour's after-stirring. Then the solid was filtered out, washed first with a mixture of 6.0 ml formic acid and 6.0 ml distilled water and then with 10.0 ml tetrahydrofuran, and dried at a reduced pressure of 4-6 kPa for 4 hour. 13.8 g crystalline 5-{2-[4-(1,2-benzisothiazol-3-yl)-1-piperazinyl]-ethyl}-6-chloro-1,3-dihydro-2H-indol-2-one-hydrobromide monohydrate (ziprasidone-hydrobromide monohydrate) in a form of Ziprasidone-hydrobromide Form I was obtained.

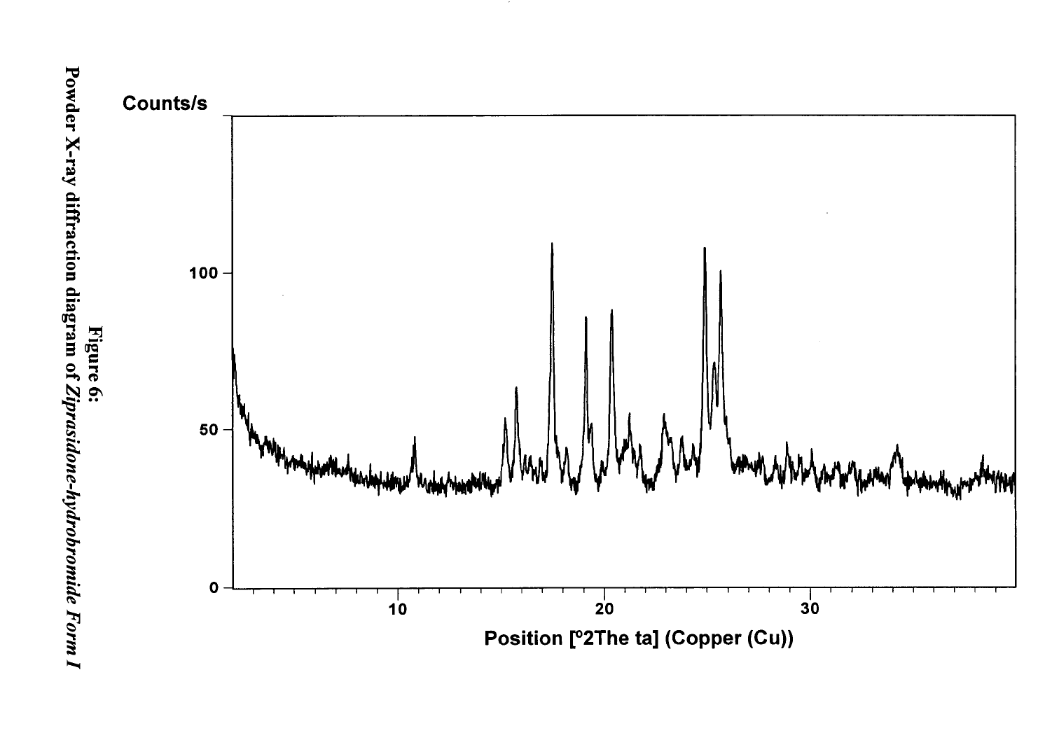

Powder X-ray diffraction diagram of the product is shown in FIG. 6, the characteristic 2θ values are: 10.834, 15.746, 17.486, 19.138, 20.383, 24.906 and 25.673 [°]. The water content determined by titrimetry with Karl Fischer method: 3.72%.

### Example 2

### Preparation of Ziprasidone-hydrobromide Form I

3.0 g 5-{2-[4-(1,2-benzisothiazol-3-yl)-1 -piperazinyl]-ethyl}-6-chloro-1,3 -dihydro-2H-indol-2-one (ziprasidone base) was dissolved in 12.0 ml formic acid at room temperature. The homogeneous solution was stirred with 0.2 g charcoal and 0.2 g silica gel 60 (particle size 0.040-0.063 mm) for 30 min, then it was filtered. The clear filtered solution was added dropvise, with stirring, in one hour into a mixture of 3.0 ml aqueous 48% (w/v) hydrogen bromide solution and 27.0 ml isopropanol at 25-30° C. temperature, followed by 1 hour's after-stirring. Then the solid was filtered out, washed first with a mixture of 3.0 ml formic acid and 3.0 ml isopropanol and then with 3.0 ml isopropanol, and dried at a reduced pressure of 4-6 kPa for 4 hour.

3.32 g crystalline 5-{2-[4-(1,2-benzisothiazol-3-yl)-1-piperazinyl]-ethyl}-6-chloro-1,3-dihydro-2H-indol-2-one-hydrobromide monohydrate (ziprasidone-hydrobromide monohydrate) in a form of Ziprasidone-hydrobromide Form I was obtained.

The IR spectrum and the powder X-ray diffraction diagram of the product are basically the same as in Example 1.

### Example 3

### Preparation of Ziprasidone-hydrobromide Form I

4.0 g 5-{2-[4-(1,2-benzisothiazol-3-yl)-1-piperazinyl]-ethyl}-6-chloro-1,3-dihydro-2H-indol-2-one (ziprasidone base) was dissolved in boiling mixture of 4.0 ml distilled water and 56.0 ml tetrahydrofuran. The homogeneous solution was stirred with 0.2 g charcoal and 0.2 g silica gel 60 (particle size 0.040-0.063 mm) for 5 min, then it was filtered. 2.0 ml aqueous 48% (w/v) hydrogen bromide solution was added dropvise into the clear filtered solution at a temperature of 60-65° C., followed by an hour's after-stirring. Then the solid was filtered out, washed with 3.0 ml tetrahydrofuran, and dried at a reduced pressure of 4-6 kPa for 4 hour.

3.68 g crystalline 5-{2-[4-(1,2-benzisothiazol-3-yl)-1-piperazinyl]-ethyl}-6-chloro-1,3-dihydro-2H-indol-2-one-hydrobromide monohydrate (ziprasidone-hydrobromide monohydrate) in a form of Ziprasidone-hydrobromide Form I was obtained.

The IR spectrum and the powder X-ray diffraction diagram of the product are basically the same as in Example 1. Water content determined by Karl Fischer method: 3.49%.

### Example 4

### Preparation of Ziprasidone-hydrobromide Form I

25.0 g 5-{2-[4-(1,2-benzisothiazol-3-yl)-1-piperazinyl]-ethyl}-6-chloro-1,3-dihydro-2H-indol-2-one (ziprasidone base) was suspended at room temperature in a mixture of 12.5 ml distilled water and 112.5 ml ethanol, and then 8.2 ml 48% (w/v) hydrogen bromide solution was added. The suspension was boiled for 2 hours, and then it was cooled to room temperature, filtered, washed twice with 20.0 ml portions of ethanol, and dried under an infrared lamp.

30.1 g crystalline 5-{2-[4-(1,2-benzisothiazol-3-yl)-1-piperazinyl]-ethyl}-6-chloro-1,3-dihydro-2H-indol-2-one-hydrobromide monohydrate (ziprasidone-hydrobromide monohydrate) in a form of Ziprasidone-hydrobromide Form I was obtained. Water content determined by Karl Fischer method: 3.60%.

The IR spectrum and the powder X-ray diffraction diagram of the product are basically the same as in Example 1.

### Example 5

### Preparation of Ziprasidone-hydrobromide Form I

3.0 g crystalline 5-{2-[4-(1,2-benzisothiazol-3-yl)-1-piperazinyl]-ethyl}-6-chloro-1,3-dihydro-2H-indol-2-one hydrobromide anhydrate (ziprasidone hydrobromide anhydrate) in form of Ziprasidone-hydrobromide Form II was dissolved in 12.0 ml formic acid at a temperature of 75-80° C. 36.0 ml distilled water was added dropvise into the homogeneous solution, with stirring, in 30 min, followed by 1 hour's after-stirring. Then the solid was filtered out, washed with a mixture of 3.0 ml distilled water and 3.0 ml methanol, and dried at a reduced pressure of 4-6 kPa for 4 hour.

2.89 g crystalline 5-{2-[4-(1,2-benzisothiazol-3-yl)-1-piperazinyl]-ethyl }-6-chloro-1,3-dihydro-2H-indol-2-one-hydrobromide monohydrate (ziprasidone-hydrobromide monohydrate) in a form of Ziprasidone-hydrobromide Form I was obtained.

The IR spectrum and the powder X-ray diffraction diagram of the product are basically the same as in Example 1.

### Example 6

### Preparation of Ziprasidone-hydrobromide Form II

20.0 g 5-{2-[4-(1,2-benzisothiazol-3-yl)-1-piperazinyl]-ethyl}-6-chloro-1,3-dihydro-2H-indol-2-one (ziprasidone base) was dissolved in a mixture of 50.0 ml dimethylacetamid and 100.0 ml methanol at room temperature, and 20.0 ml aqueous 48% (w/v) hydrogen bromide was added into it. The formed suspension was boiled for 16 hour, and then was cooled back to room temperature. The solid was filtered out, washed with methanol, and dried under infrared lamp.

23.2 g crystalline 5-{2-[4-(1,2-benzisothiazol-3-yl)-1-piperazinyl]-ethyl}-6-chloro-1,3-dihydro-2H-indol-2-one-hydrobromide anhydrate (ziprasidone-hydrobromide anhydrate) in a form of Ziprasidone-hydrobromide Form II was obtained.

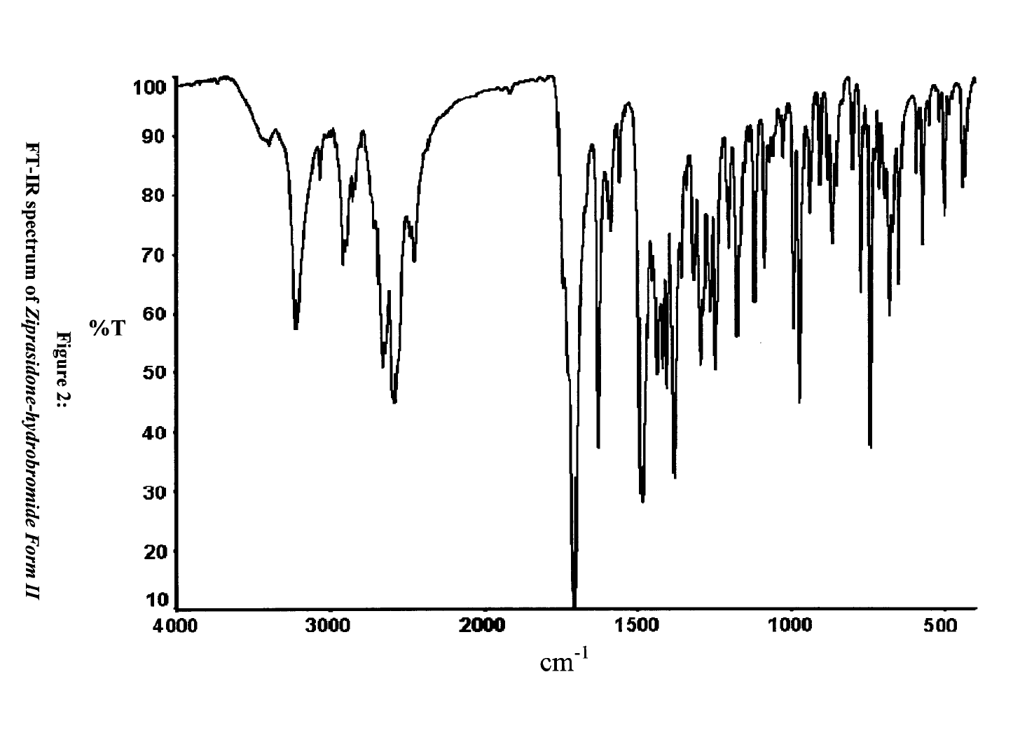

The FT-IR spectrum of the product is shown in FIG. 2. The characteristic bands are at: 3224, 2582, 1708, 1628, 1486, 973 and 905 cm⁻¹ values.

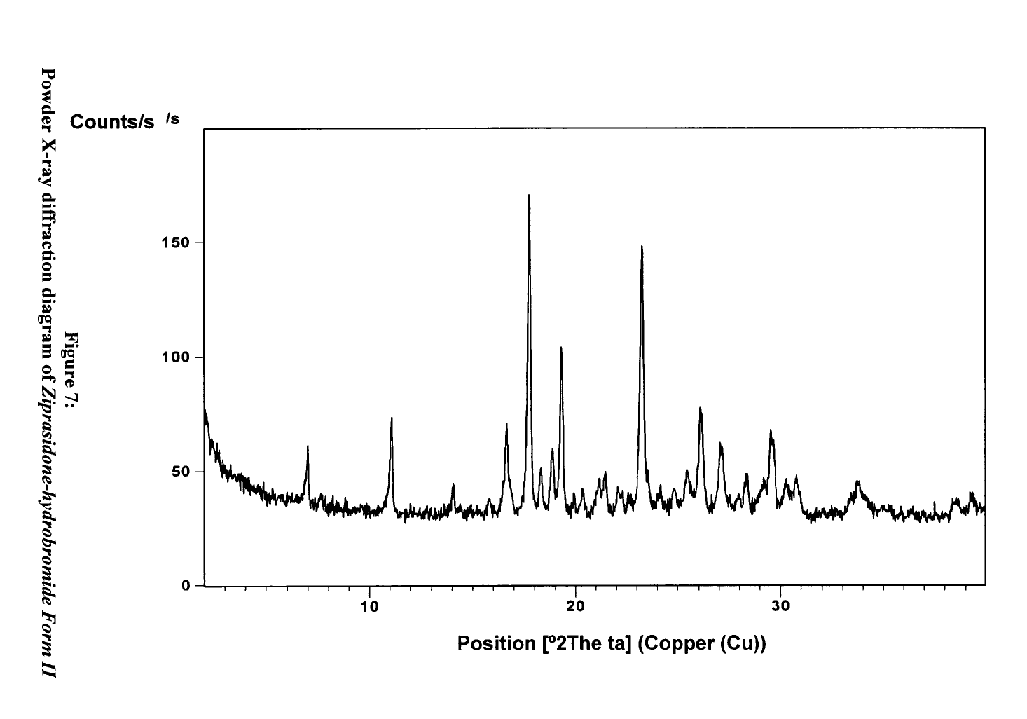

Powder X-ray diffraction diagram of the product is shown in FIG. 7, according to it the characteristic 20 values are: 7.014, 11.081, 17.759, 19.339, 23.283, 26.094 and 29.498 [°].

During the thermogravimetric investigation with heating up to 150° C. the mass loss was 0.87%.

### Example 7

### Preparation of Ziprasidone-hydrobromide Form II

2.0 g 5-{2-[4-(1,2-benzisothiazol-3-yl)-1-piperazinyl]-ethyl}-6-chloro-1,3-dihydro-2H-indol-2-one (ziprasidone base) was suspended in 30.0 ml methanol at room temperature, then 2.0 ml glacial acetic acidic 33% (w/v) hydrogen bromide solution was added. After stirring the suspension for 16 hours at room temperature the solid was filtered, washed with methanol, and dried under an infrared lamp.

2.39 g crystalline 5-{2-[4-(1,2-benzisothiazol-3-yl)-1-piperazinyl]-ethyl}-6-chloro-1,3-dihydro-2H-indol-2-one-hydrobromide anhydrate (ziprasidone-hydrobromide anhydrate) in the form of Ziprasidone-hydrobromide Form II was obtained.

The IR spectrum and the powder X-ray diffraction diagram of the product are basically the same as in Example 6.

### Example 8

### Preparation of Ziprasidone-hydrobromide Form II

2.0 g 5-{2-[4-(1,2-benzisothiazol-3-yl)-1-piperazinyl]-ethyl}-6-chloro-1,3-dihydro-2H-indol-2-one (ziprasidone base) was suspended in 30.0 ml methanol at room temperature, then 2.0 ml aqueous 48% (w/v) hydrogen bromide solution was added. After stirring the suspension for 16 hours at room temperature the solid was filtered, washed with methanol, and dried under an infrared lamp.

2.39 g crystalline 5-{2-[4-(1,2-benzisothiazol-3-yl)-1-piperazinyl]-ethyl}-6-chloro-1,3-dihydro-2H-indol-2-one-hydrobromide anhydrate (ziprasidone-hydrobromide anhydrate) in a form of Ziprasidone-hydrobromide Form II was obtained.

The IR spectrum and the powder X-ray diffraction diagram of the product are basically the same as in Example 6.

### Example 9

### Preparation of Ziprasidone-hydrobromide Form II

2.0 g 5-{2-[4-(1,2-benzisothiazol-3-yl)-1-piperazinyl]-ethyl}-6-chloro-1,3-dihydro-2H-indol-2-one (ziprasidone base) was suspended in 20.0 ml tetrahydrofuran at room temperature, then with continuous stirring, 2.0 ml glacial acetic acidic 33% (w/v) hydrogen bromide solution was added. The suspension was stirred for 16 hours at a temperature of 60-65° C., after cooling back to room temperature the solid was filtered, washed with methanol, and dried under an infrared lamp.

2.38 g crystalline 5-{2-[4-(1,2-benzisothiazol-3-yl)-1-piperazinyl]-ethyl}-6-chloro-1,3-dihydro-2H-indol-2-one-hydrobromide anhydrate (ziprasidone-hydrobromide anhydrate) in a form of Ziprasidone-hydrobromide Form II was obtained.

The IR spectrum and the powder X-ray diffraction diagram of the product are basically the same as in Example 6.

During the thermogravimetric investigation with heating up to 150° C. the mass loss was 0.36%.

### Example 10

### Preparation of Ziprasidone-hydrobromide Form II

2.0 g 5-{2-[4-(1,2-benzisothiazol-3-yl)-1-piperazinyl]-ethyl}-6-chloro-1,3-dihydro-2H-indol-2-one (ziprasidone base) was dissolved in a boiling mixture of 20.0 ml ethanol and 10.0 ml glacial acetic acid, after cooling it was filtered to fiber-free, and 2.0 ml glacial acetic acidic 33% (w/v) hydrogen bromide solution was added into it. The formed suspension was boiled for 16 hour, and then was cooled back to room temperature. The solid was filtered out, washed with methanol, and dried under infrared lamp.

2.12 g crystalline 5-{2-[4-(1,2-benzisothiazol-3-yl)-1-piperazinyl]-ethyl}-6-chloro-1,3 -dihydro-2H-indol-2-one-hydrobromide anhydrate (ziprasidone-hydrobromide anhydrate) in a form of Ziprasidone-hydrobromide Form II was obtained.

The IR spectrum and the powder X-ray diffraction diagram of the product are basically the same as in Example 6.

During the thermogravimetric investigation with heating up to 150° C. the mass loss was 0.58%.

### Example 11

### Preparation of Ziprasidone-hydrobromide Form II

2.0 g 5-{2-[4-(1,2-benzisothiazol-3-yl)-1-piperazinyl]-ethyl}-6-chloro-1,3-dihydro-2H-indol-2-one hydrobromide monohydrate (ziprasidone hydrobromide monohydrate) was dissolved in 6.0 ml formic acid at a temperature of 90-95° C. This solution was added dropvise, with stirring, in 1 min into 40.0 ml methyl tertiary-butyl ether at room temperature. The solid was filtered out from the formed suspension, was washed with methyl tertiary-butyl ether and dried under infrared lamp.

1.87 g crystalline 5-{2-[4-(1,2-benzisothiazol-3-yl)-1-piperazinyl]-ethyl}-6-chloro-1,3-dihydro-2H-indol-2-one-hydrobromide anhydrate (ziprasidone-hydrobromide anhydrate) in a form of Ziprasidone-hydrobromide Form II was obtained.

The IR spectrum and the powder X-ray diffraction diagram of the product are basically the same as in Example 6.

During the thermogravimetric investigation with heating up to 150° C. the loss was 0.63%.

### Example 12

### Preparation of Ziprasidone-hydrobromide Form II

2.0 g 5-{2-[4-(1,2-benzisothiazol-3-yl)-1-piperazinyl]-ethyl}-6-chloro-1,3-dihydro-2H-indol-2-one hydrobromide hemihydrate (ziprasidone hydrobromide hemihydrate) in form of Ziprasidone-hydrobromide Form III was dissolved in 6.0 ml formic acid at a temperature of 90-95° C. This solution was added dropvise, with stirring, in 1 min into 20.0 ml methyl tertiary-butyl ether at a temperature of 5-10° C., followed by 30 min. after-stirring, and then the solid was filtered out, washed with 2.0 ml methyl tertiary-butyl ether, and dried at a reduced pressure of 4-6 kPa for 4 hour.

1.85 g crystalline 5-{2-[4-(1,2-benzisothiazol-3-yl)-1-piperazinyl]-ethyl}-6-chloro-1,3-dihydro-2H-indol-2-one-hydrobromide anhydrate (ziprasidone-hydrobromide anhydrate) in a form of Ziprasidone-hydrobromide Form II was obtained.

The IR spectrum and the powder X-ray diffraction diagram of the product are basically the same as in Example 6.

During the thermogravimetric investigation with heating up to 150° C. the mass loss was 0.69%.

### Example 13

### Preparation of Ziprasidone-hydrobromide Form II

2.0 g 5-{2-[4-(1,2-benzisothiazol-3-yl)-1-piperazinyl]-ethyl}-6-chloro-1,3-dihydro-2H-indol-2-one (ziprasidone base) was dissolved in a boiling mixture of 20.0 ml ethanol and 10.0 ml glacial acetic acid. The homogeneous solution was stirred with 0.2 g charcoal and 0.2 g silica gel 60 (particle size 0.040-0.063 mm) for 5 min, then it was filtered. 2.0 ml aqueous 48% (w/v) hydrogen bromide solution was added into the clear filtered solution, the formed suspension was boiled for 1 hour, then it was cooled back to room temperature, followed by an hour's after-stirring. Then the solid was filtered out, washed twice with 2.0 ml portions ethanol, and dried at a reduced pressure of 4-6 kPa for 4 hour.

2.24 g crystalline 5-{2-[4-(1,2-benzisothiazol-3-yl)-1-piperazinyl]-ethyl}-6-chloro-1,3-dihydro-2H-indol-2-one-hydrobromide anhydrate (ziprasidone-hydrobromide anhydrate) in a form of Ziprasidone-hydrobromide Form II was obtained.

The IR spectrum and the powder X-ray diffraction diagram of the product are basically the same as in Example 6.

During the thermogravimetric investigation with heating up to 150° C. the mass loss was 0.62%.

### Example 14

### Preparation of Ziprasidone-hydrobromide Form II

2.0 g 5-{2-[4-(1,2-benzisothiazol-3-yl)-1-piperazinyl]-ethyl}-6-chloro-1,3-dihydro-2H-indol-2-one (ziprasidone base) was dissolved in a boiling mixture of 63.0 ml tetrahydrofuran and 7.0 ml glacial acetic acid. The homogeneous solution was stirred with 0.2 g charcoal and 0.2 g silica gel 60 (particle size 0.040-0.063 mm) for 5 min, then it was filtered. 2.0 ml glacial acetic acidic 33% (w/v) hydrogen bromide solution was added into the clear filtered solution, the formed suspension was boiled for 1 hour, then it was cooled back to room temperature, followed by an hour's after-stirring. Then the solid was filtered out, washed twice with 2.0 ml portions of tetrahydrofuran, and dried at a reduced pressure of 4-6 kPa for 4 hour.

2.30 g crystalline 5-{2-[4-(1,2-benzisothiazol-3-yl)-1-piperazinyl]-ethyl}-6-chloro-1,3-dihydro-2H-indol-2-one-hydrobromide anhydrate (ziprasidone-hydrobromide anhydrate) in a form of Ziprasidone-hydrobromide Form II was obtained.

The IR spectrum and the powder X-ray diffraction diagram of the product are basically the same as in Example 6.

During the thermogravimetric investigation with heating up to 150° C. the mass loss was 0.62%.

### Example 15

### Preparation of Ziprasidone-hydrobromide Form II

2.0 g 5-{2-[4-(1,2-benzisothiazol-3-yl)-1-piperazinyl]-ethyl}-6-chloro-1,3-dihydro-2H-indol-2-one sesquihydrobromide hemiformiate (sesquihydrobromide hemiformiate) in the form of Ziprasidone hydrobromide Form IV prepared according to Example 18 was heated to temperature in the range of 180-200° C., then it was cooled back to room temperature.

1.69 g crystalline 5-{2-[4-(1,2-benzisothiazol-3-yl)-1-piperazinyl]-ethyl}-6-chloro-1,3-dihydro-2H-indol-2-one-hydrobromide anhydrate (ziprasidone-hydrobromide anhydrate) in a form of Ziprasidone-hydrobromide Form II was obtained. This product does not absorb water from the air in normal circumstances.

The IR spectrum and the powder X-ray diffraction diagram of the product are basically the same as in Example 6.

### Example 16

### Preparation of Ziprasidone-hydrobromide Form III

18.0 g 5-{2-[4-(1,2-benzisothiazol-3-yl)-1-piperazinyl]-ethyl}-6-chloro-1,3-dihydro-2H-indol-2-one (ziprasidone base) was dissolved in 72.0 ml formic acid at room temperature. The homogeneous solution was stirred with 1.2 g charcoal and 1.2 g silica gel 60 (particle size 0.040-0.063 mm) for 30 min, then it was filtered. A mixture of 6.0 ml aqueous 48% (w/v) hydrogen bromide solution and 64.0 ml distilled water was added dropvise, with stirring, into the clear filtered solution in 1 hour, followed by 1 hour's after-stirring. Then the solid was filtered out, washed first with a mixture of 10 ml distilled water and 10 ml formic acid then with 20.0 ml tetrahydrofuran, and dried at a reduced pressure of 4-6 kPa for 4 hour.

20.9 g crystalline 5-{2-[4-(1,2-benzisothiazol-3-yl)-1-piperazinyl]-ethyl}-6-chloro-1,3 -dihydro-2H-indol-2-one-hydrobromide hemihydrate (ziprasidone-hydrobromide hemihydrate) in a form of Ziprasidone-hydrobromide Form III was obtained.

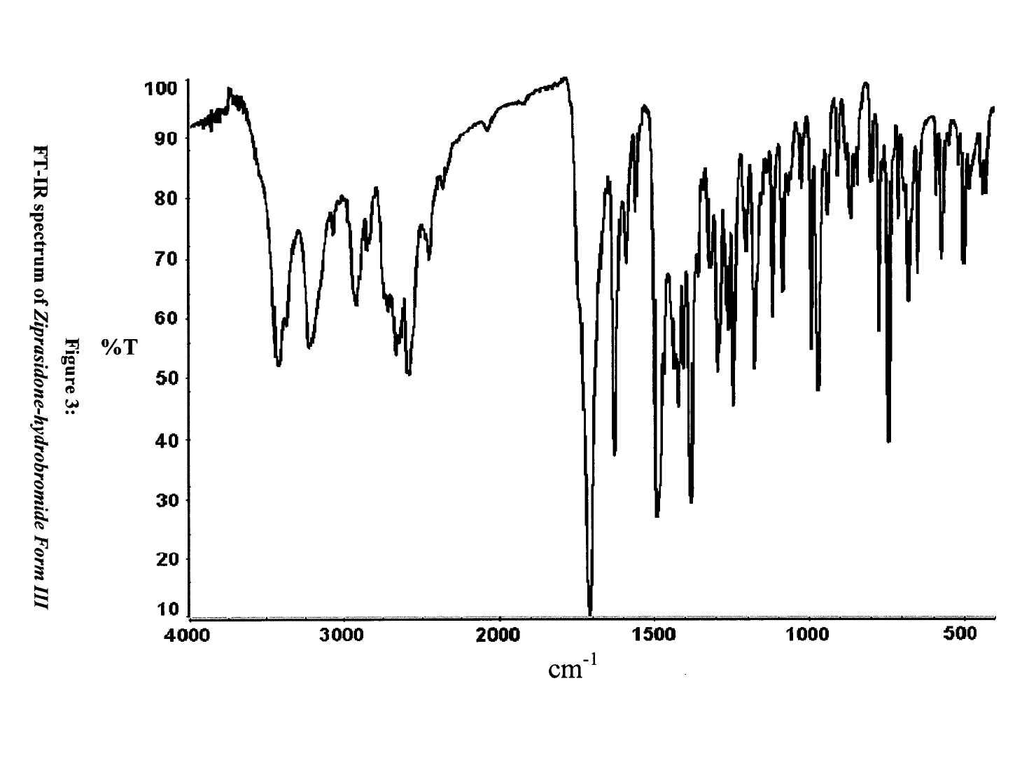

FT-IR spectrum of the product was shown in FIG. 3. The characteristic bands are at: 3423, 3223, 2917, 1710, 1494, 972 and 741 cm⁻¹ values.

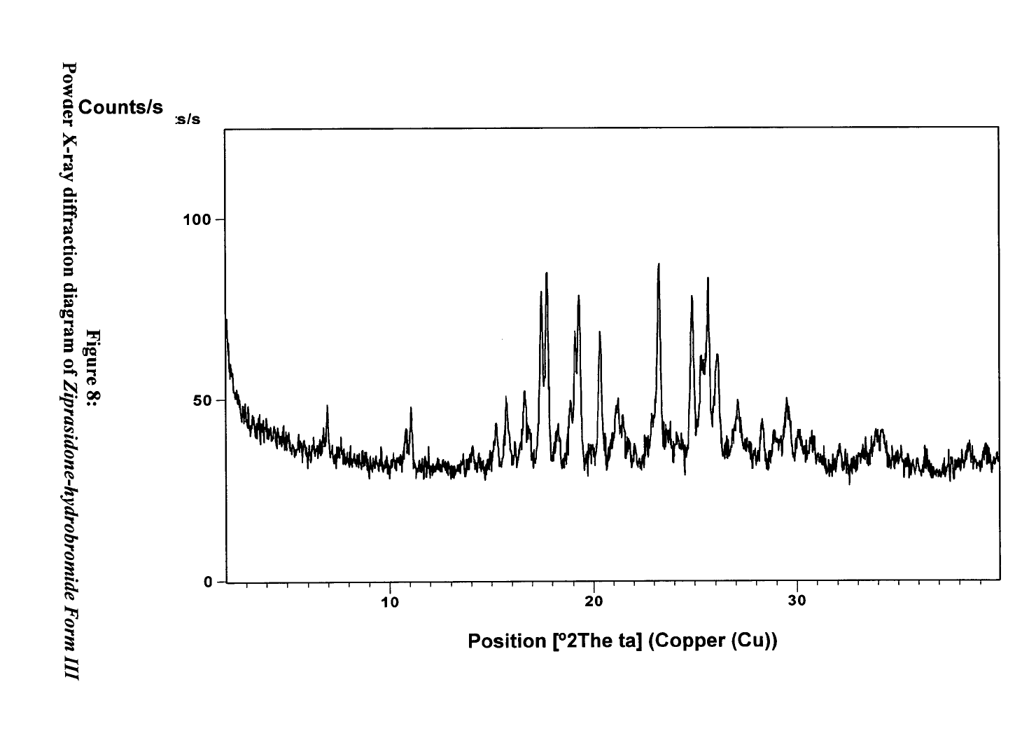

Powder X-ray diffraction diagram of the product was shown in FIG. 8, according to it the characteristic 20 values are: 6,986, 11,068, 17,468, 17,744, 19,319, 23,247 and 25,661 [°]

During the thermogravimetric investigation with heating up to 150° C. the mass loss was 1.67%.

### Example 17

### Preparation of Ziprasidone-hydrobromide Form III

2.0 g 5-{2-[4-(1,2-benzisothiazol-3-yl)-1-piperazinyl]-ethyl}-6-chloro-1,3-dihydro-2H-indol-2-one hydrobromide monohydrate (ziprasidone hydrobromide monohydrate) in the form of Ziprasidone hydrobromide Form I was dissolved in 6.0 ml formic acid at a temperature of 90-95° C. This solution was added dropvise, with stirring, in 1 min into 20.0 ml distilled water at a temperature of 90-95° C. followed by 30 min after-stirring. The solid was filtered out, washed first with 2.0 ml distilled water, then with 2.0 ml methanol, and dried at a reduced pressure of 4-6 kPa for 4 hour.

1.95 g crystalline 5-{2-[4-(1,2-benzisothiazol-3-yl)-1-piperazinyl]-ethyl}-6-chloro-1,3-dihydro-2H-indol-2-one-hydrobromide hemihydrate (ziprasidone-hydrobromide hemihydrate) in a form of Ziprasidone-hydrobromide Form III was obtained.

The IR spectrum and the powder X-ray diffraction diagram of the product are basically the same as in Example 16.

### Example 18

### Preparation of Ziprasidone-hydrobromide Form IV

2.0 g 5-{2-[4-(1,2-benzisothiazol-3-yl)-1-piperazinyl]-ethyl}-6-chloro-1,3-dihydro-2H-indol-2-one (ziprasidone base) was dissolved in 8.0 ml formic acid at room temperature. The homogeneous solution was stirred with 0.2 g charcoal and 0.2 g silica gel 60 (particle size 0.040-0.063 mm) for 30 min, then it was filtered. A mixture of 2.0 ml aqueous 48% (w/v) hydrogen bromide solution and 10.0 ml methyl-isobutyl ketone was added dropvise, with stirring, in 15 min into the clear filtered solution at a temperature of 25-30° C., followed by an overnight's after-stirring. Then the solid was filtered out, washed with 3.0 ml methyl-isobutyl ketone, and dried under infrared lamp.

2.71 g crystalline 5-{2-[4-(1,2-benzisothiazol-3-yl)-1-piperazinyl]-ethyl}-6-chloro-1,3-dihydro-2H-indol-2-one sesquihydrobromide hemiformiate (ziprasidone sesquihydrobromide hemi-formiate) in a form of Ziprasidone-hydrobromide Form IV was obtained.

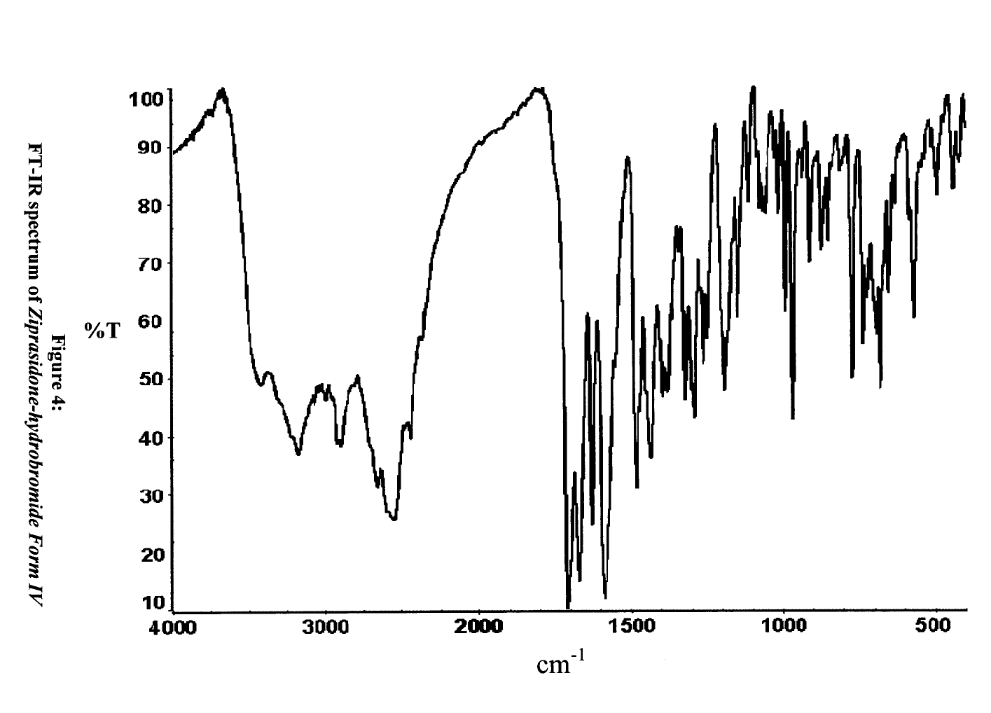

FT-IR spectrum of the product was shown in FIG. 4. The characteristic bands are at: 3423, 3223, 2917, 1710, 1494, 972 and 741 cm⁻¹ values.

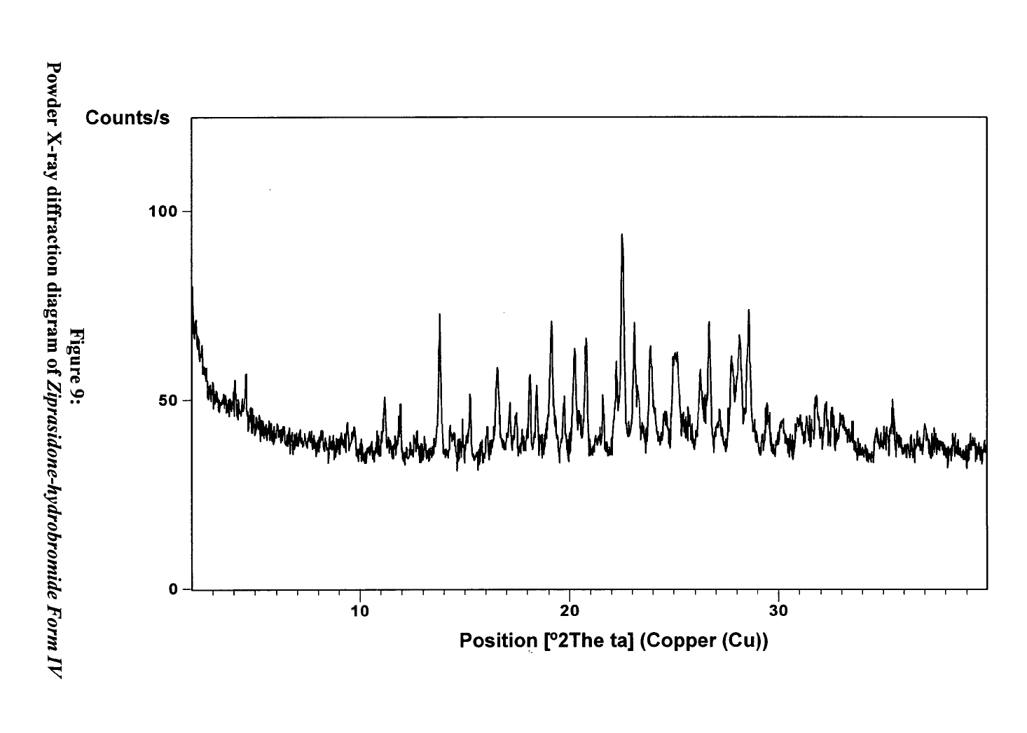

Powder X-ray diffraction diagram of the product is shown in FIG. 9, according to it the characteristic 20 values are: 6,986, 11,068, 17,468, 17,744, 19,319,23,247 and 25,661 [°].

The water content determined with Karl Fischer method: 4.88 %(w/w). The molar contents of HBr and formic acid calculated to the ziprasidone base, and determined by a potentiometric titrimetric and an NMR method: 1.52 (m/m) and 0.62 (m/m), respectively.

### Example 19

### Preparation of Ziprasidone-hydrobromide Form IV

2.0 g 5-{2-[4-(1,2-benzisothiazol-3-yl)-1-piperazinyl]-ethyl}-6-chloro-1,3-dihydro-2H-indol-2-one (ziprasidone base) was dissolved in 10.0 ml formic acid at room temperature. The homogeneous solution was stirred with 0.2 g charcoal and 0.2 g silica gel 60 (particle size 0.040-0.063 mm) for 30 min, then it was filtered. A mixture of 2.0 ml glacial acetic acidic 33% (w/v) hydrogen bromide solution and 10.0 ml ethyl acetate was added dropvise, with stirring, in 15 min into the clear filtered solution at a temperature of 25-30° C., followed by an overnight's after-stirring. Then the solid was filtered out, washed with 3.0 ml ethyl acetate, and dried under infrared lamp.

2.23 g crystalline 5-{2-[4-(1,2-benzisothiazol-3-yl)-1-piperazinyl]-ethyl}-6-chloro-1,3-dihydro-2H-indol-2-one sesquihydrobromide hemiformiate (ziprasidone sesquihydrobromide hemi-formiate) in a form of Ziprasidone-hydrobromide Form IV was obtained.

The IR spectrum and the powder X-ray diffraction diagram of the product are basically the same as in Example 18.

The water content determined with Karl Fischer method: 2.91% (w/w). The molar contents of HBr and formic acid calculated to the ziprasidone base, and determined by a potentiometric titrimetric and an NMR method: 1.56 (m/m) and 0.80 (m/m), respectively.

### Example 20

### Preparation of Ziprasidone-hydrobromide Form IV

2.0 g 5-{2-[4-(1,2-benzisothiazol-3-yl)-1-piperazinyl]-ethyl}-6-chloro-1,3-dihydro-2H-indol-2-one (ziprasidone base) was dissolved in 8.0 ml formic acid at room temperature. The homogeneous solution was stirred with 0.2 g charcoal and 0.2 g silica gel 60 (particle size 0.040-0.063 mm) for 30 min, then it was filtered. A mixture of 2.0 ml aqueous 48% (w/v) hydrogen bromide solution and 10.0 ml tetrahydrofuran was added dropvise into the clear filtered solution, with stirring at a temperature of 25-30° C. in 15 min, followed by an overnight's after-stirring. Then the solid was filtered out, washed with 3.0 ml tetrahydrofuran, and dried under infrared lamp.

2.87 g crystalline 5-{2-[4-(1,2-benzisothiazol-3-yl)-1-piperazinyl]-ethyl}-6-chloro-1,3-dihydro-2H-indol-2-one sesquihydrobromide hemiformiate (ziprasidone sesquihydrobromide hemi-formiate) in a form of Ziprasidone-hydrobromide Form IV was obtained.

The IR spectrum and the powder X-ray diffraction diagram of the product are basically the same as in Example 18.

The water content determined with Karl Fischer method: 5.28% (w/w). The molar contents of HBr and formic acid calculated to the ziprasidone base, and determined by a potentiometric titrimetric and an NMR method: 1.67 (m/m) and 0.59 (m/m), respectively.

### Example 21

### Preparation of Ziprasidone-hydrobromide Form IV

2.0 g 5-{2-[4-(1,2-benzisothiazol-3-yl)-1-piperazinyl]-ethyl}-6-chloro-1,3-dihydro-2H-indol-2-one (ziprasidone base) was dissolved in 8.0 ml formic acid at room temperature. The homogeneous solution was stirred with 0.2 g charcoal and 0.2 g silica gel 60 (particle size 0.040-0.063 mm) for 30 min, then it was filtered. A mixture of 2.0 ml aqueous 48% (w/v) hydrogen bromide solution and 10.0 ml ethyl acetate was added dropvise into the clear filtered solution, with stirring at a temperature of 25-30° C. in 15 min, followed by an overnight's after-stirring. Then the solid was filtered out, washed with 3.0 ml ethyl acetate, and dried under infrared lamp.

2.74 g crystalline 5-{2-[4-(1,2-benzisothiazol-3-yl)-1-piperazinyl]-ethyl}-6-chloro-1,3-dihydro-2H-indol-2-one sesquihydrobromide hemiformiate (ziprasidone sesquihydrobromide hemi-formiate) in a form of Ziprasidone-hydrobromide Form IV was obtained.

The IR spectrum and the powder X-ray diffraction diagram of the product are basically the same as in Example 18.

The water content determined with Karl Fischer method: 4.02% (w/w). The molar contents of HBr and formic acid calculated to the ziprasidone base, and determined by a potentiometric titrimetric and an NMR method: 1.46 (m/m) and 0.57 (m/m), respectively.

### Example 22

### Preparation of Ziprasidone-hydrobromide Form V

2.0 g 5-{2-[4-(1,2-benzisothiazol-3-yl)-1-piperazinyl]-ethyl}-6-chloro-1,3-dihydro-2H-indol-2-one (ziprasidone base) was dissolved in 10.0 ml formic acid at room temperature. The homogeneous solution was stirred with 0.2 g charcoal and 0.2 g silica gel 60 (particle size 0.040-0.063 mm) for 30 min, then it was filtered. A mixture of 2.0 ml glacial acetic acidic 33% (w/v) hydrogen bromide solution and 10.0 ml methyl isobutyl ketone was added, with stirring, in 1 min into the clear filtered solution, at a temperature of 65-70° C., followed by an overnight's after-stirring. Then the solid was filtered out, washed with 3.0 ml methyl isobutyl ketone, and dried under infrared lamp.

2.38 g amorphous 5-{2-[4-(1,2-benzisothiazol-3-yl)-1-piperazinyl]-ethyl}-6-chloro-1,3-dihydro-2H-indol-2-one hydrobromide (ziprasidone hydrobromide) in a form of Ziprasidone-hydrobromide Form V was obtained.

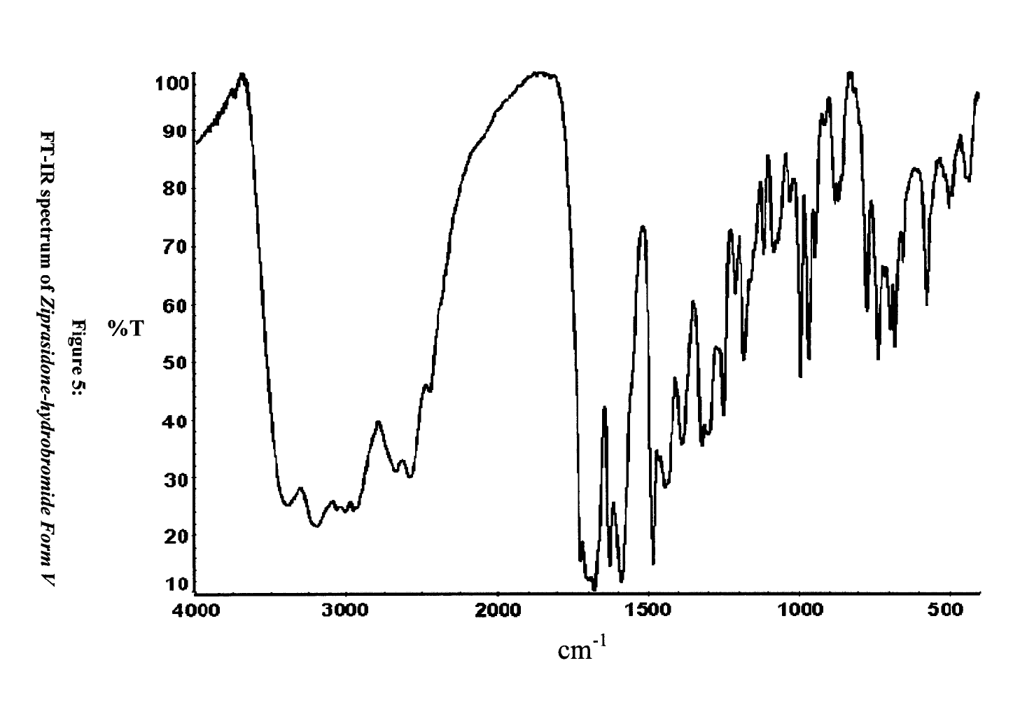

FT-IR spectrum of the product is shown in FIG. 5. The characteristic bands are at: 3410, 2808, 1723, 1156, 820, 770 and 736 cm⁻¹ values.

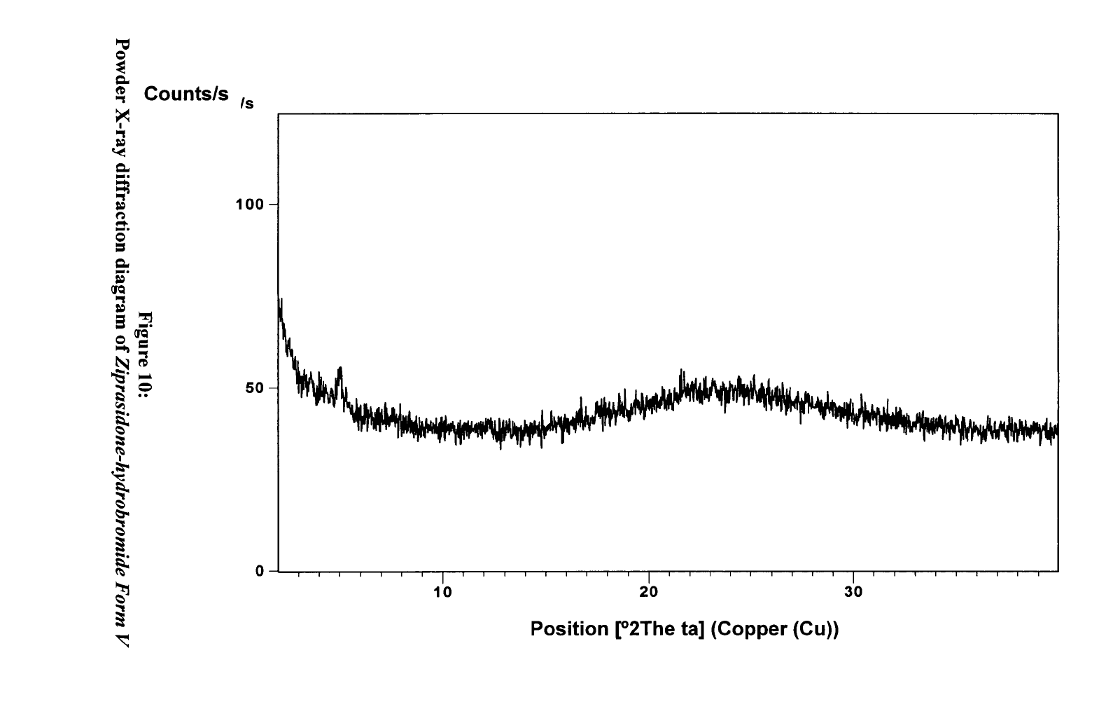

Powder X-ray diffraction diagram of the product is shown in FIG. 10, on which lacks of reflection maxima according to the characteristics of this product.

The water content determined by a Karl Fischer titrimetric method: 5.62%.

1. A compound or polymorph form selected from the group consiting of:
Ziprasidone-hydrobromide monohydrate, that is, 5-{2-[4-(1,2-benzisothiazol-3-yl)-1-piperazinyl]-ethyl}-6-chloro-1,3-dihydro-2H-indol-2-one hydrobromide monohydrate;
Ziprasidone-hydrobromide Form I, a crystalline morphological modification of the ziprasidone-hydrobromide monohydrate characterized by infrared spectrum bands at 3427, 3369, 2937, 2598, 1713, 1494, 968 and 843 cm⁻¹±4 cm⁻¹, and further characterized by Powder X-ray diffraction peaks at 10.8, 15.7, 17.5, 19.1, 20.4, 24.9 and 25.7 [°] 10.2 [°] 2θ diffraction angles;
crystalline ziprasidone-hydrobromide anhydrate, that is, crystalline 5-{2-[4-(1,2-benzisothiazol-3-yl) -1-piperazinyl]-ethyl}-6-chloro-1,3-dihydro-2H-indol-2-one hydrobromide anhydrate;
Ziprasidone-hydrobromide Form II, a crystalline morphological modification of the crystalline ziprasidone-hydrobromide anhydrate characterized by infrared spectrum bands at 3224, 2582, 1708, 1628, 1486, 973 and 905 cm⁻¹±4 cm⁻¹, and further characterized by powder X-ray diffraction peaks at 7.0, 11.1, 17.8, 19.3, 23.3, 26.1 and 29.5 [°]±0.2 [°] 2θ diffraction angles;
ziprasidone-hydrobromide hemihydrate, that is, 5-{2-[4-(1,2-benzisothiazol-3-yl)-1-piperazinyl]-ethyl}-6-chloro-1,3-dihydro-2H-indol-2-one hydrobromide hemihydrate;
Ziprasidone-hydrobromide Form III, crystalline morphological modification of the compound of the ziprasidone-hydrobromide hemihydrate characterized by infrared spectrum bands at 3423, 3223, 2917, 1710, 1494, 972 and 741 cm⁻¹±4 cm⁻¹, and further characterized by Powder X-ray diffraction peaks at 7.0, 11.1, 17.5, 17.7, 19.3, 23.2 and 25.7 [°] 0.2 [°] 2θ diffraction angles;
ziprasidone-sesquihydrobromide hemiformiate, that is, 5-{2-[4-(1,2-benzisothiazol-3-yl)-1-piperazinyl]-ethyl}-6-chloro-1,3-dihydro-2H-indol-2-one sesquihydrobromide hemiformiate;
Ziprasidone-hydrobromide Form IV, crystalline morphological modification of the compound of the ziprasidone-sesquihydrobromide hemiformiate characterized by infrared spectrum bands at 3420, 3176, 2548, 1710, 1671, 1629, 1585, 774 and 569 cm⁻¹±4 cm⁻¹, and further characterized by Powder X-ray diffraction peaks at 13.8, 16.6, 19.2, 20.8, 223, 23.1 and 28.6 [°]±0.2 [°] 2θ diffraction angles;
amorphous ziprasidone-hydrobromide, that is, amorphous 5-{2-[4-(1,2-benzisothiazol-3-yl) -1-piperazinyl]-ethyl}-6-chloro-1,3-dihydro-2H-indol-2-one hydrobromide; and
Ziprasidone-hydrobromide Form V, amorphous form of the amorphous ziprasidone-hydrobromide characterized by infrared spectrum bands at 3410, 2808, 1723, 1156, 820, 770 and 736 cm⁻¹±4 cm⁻¹, and with the lack of diffraction peaks in its powder X-ray diffractogram.

2. The compound or polymorph form of claim 1 which is Ziprasidone-hydrobromide Form I, a crystalline morphological modification of the ziprasidone-hydrobromide monohydrate characterized by infrared spectrum bands at 3427, 3369, 2937, 2598, 1713, 1494, 968 and 843 cm⁻¹±4 cm⁻¹, and further characterized by Powder X-ray diffraction peaks at 10.8, 15.7, 17.5, 19.1, 20.4, 24.9 and 25.7 [°] 10.2 [°] 2θ diffraction angles.

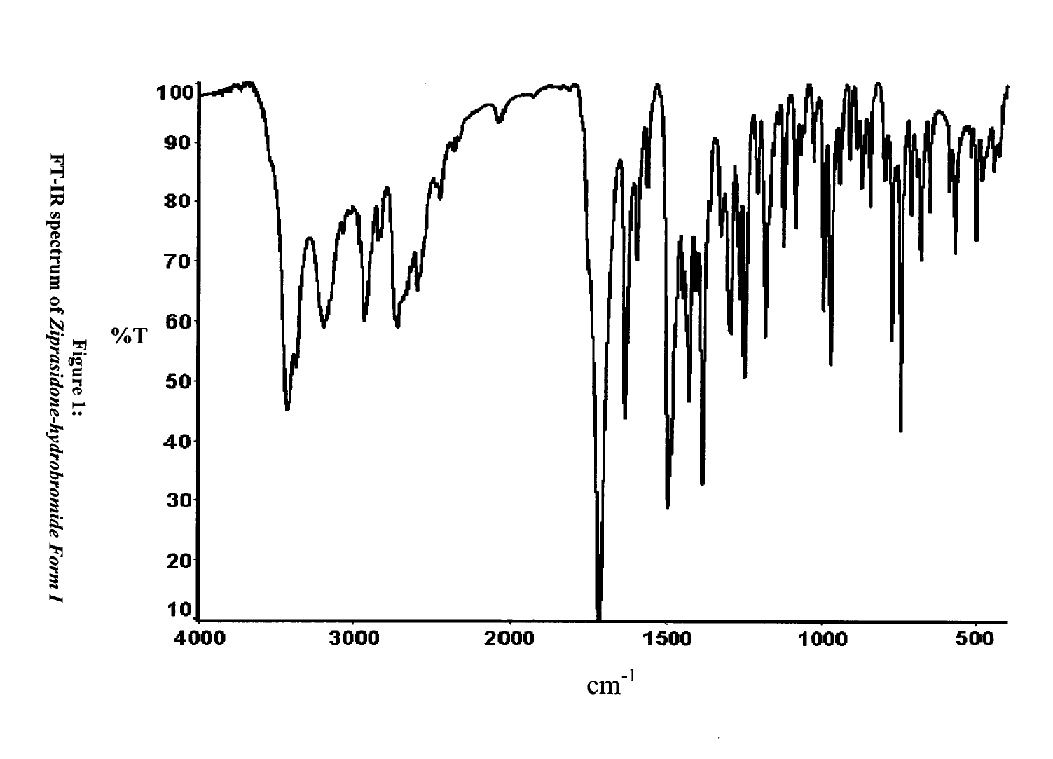

3. The compound or polymorph form of claim 2 characterized by infrared spectrum substantially depicted as in FIG. 1 and further characterized by powder X-ray diffraction substantially depicted as in FIG. 6.

4. The compound or polymorph form of claim 1 which is ziprasidone-hydrobromide monohydrate, that is, 5-{2-[4-(1,2-benzisothiazol-3-yl)-1-piperazinyl]-ethyl}-6-chloro-1,3-dihydro-2H-indol-2-one hydrobromide monohydrate, having water content of 3.0-4.0% (w/w).

5. The compound or polymorph form of claim 1 which is Crystalline ziprasidone-hydrobromide anhydrate, that is, crystalline 5-{2-[4-(1,2-benzisothiazol-3-yl)-1-piperazinyl]-ethyl }-6-chloro-1,3-dihydro-2H-indol-2-one hydrobromide anhydrate.

6. The compound or polymorph form of claim 1 which is Ziprasidone-hydrobromide Form II, a crystalline morphological modification of the crystalline ziprasidone-hydrobromide anhydrate, characterized by infrared spectrum bands at 3224, 2582, 1708, 1628, 1486, 973 and 905 cm^{31 1}±4 cm⁻¹, and further characterized by powder X-ray diffraction peaks at 7.0, 11.1, 17.8, 19.3, 23.3, 26.1 and 29.5 [°]±0.2 [°] 2θ diffraction angles.

7. The compound or polymorph form of claim 6 characterized by infrared spectrum substantially depicted as in FIG. 2 and further characterized by powder X-ray diffraction diagram substantially depicted as in FIG. 7.

8. The compound or polymorph form of claim 5 having water content less than 1.0% (w/w) determined by thermogravimetry.

9. The compound or polymorph form of claim 1 which is Ziprasidone-hydrobromide hemihydrate, that is, 5-{2-[4-(1,2-benzisothiazol-3-yl)-1-piperazinyl]-ethyl}-6-chloro-1,3-dihydro-2H-indol-2-one hydrobromide hemihydrate.

10. The compound or polymorph form of claim 1 which is Ziprasidone-hydrobromide Form III, crystalline morphological modification of the ziprasidone-hydrobromide hemihydrate characterized by infrared spectrum bands at 3423, 3223, 2917, 1710, 1494, 972 and 741 cm⁻¹4 cm⁻¹, and further characterized by Powder X-ray diffraction peaks at 7.0, 11.1, 17.5, 17.7, 19.3, 23.2 and 25.7 [°] 0.2 [°] 2θ diffraction angles.

11. The compound or polymorph form of claim 10 characterized by infrared spectrum substantially depicted as in FIG. 3 and further characterized by powder X-ray diffraction diagram substantially depicted as in FIG. 8.

12. The compound or polylmorph form of claim 9 having water content of 1.2-2.8% (w/w) determined by Karl Fischer titrimetric method.

13. The compound or polymorph form of claim 1 which is Ziprasidone-sesquihydrobromide hemiformiate, that is, 5-{2-[4-(1,2-benzisothiazol-3-yl)-1-piperazinyl]-ethyl}-6-chloro-1,3-dihydro-2H-indol-2-one sesquihydrobromide hemiformiate.

14. The compound or polymorph form of claim 1 which is Ziprasidone-hydrobromide Form IV, crystalline morphological modification of the ziprasidone-sesquihydrobromide hemiformiate characterized by infrared spectrum bands at 3420, 3176, 2548, 1710, 1671, 1629, 1585, 774 and 569 cm⁻¹±4 cm⁻¹, and further characterized by Powder X-ray diffraction peaks at 13.8, 16.6, 19.2, 20.8, 223, 23.1 and 28.6 [°]±0.2 [°] 2θ diffraction angles.

15. The compound or polymorph form of claim 14 characterized by infrared spectrum substantially depicted as in FIG. 4 and further characterized by powder X-ray diffraction diagram substantially depicted as in FIG. 9.

16. The compound or polymorph form of claim 13 having a molar ratio of hydrogen bromide content relatively to ziprasidone base of 1.3-1.7, and having a molar ratio of formic acid content relatively to ziprasidone base of 0.4-0.8.

17. The compound or polymorph form of claim 1 which is Amorphous ziprasidone-hydrobromide, that is, amorphous 5-{2-[4-(1,2-benzisothiazol-3-yl)-1-piperazinyl]-ethyl}-6-chloro-1,3-dihydro-2H-indol-2-one hydrobromide.

18. The compound or polymorph form of claim 1 which is Ziprasidone-hydrobromide Form V, amorphous form of the amorphous ziprasidone-hydrobromide characterized by infrared spectrum bands at 3410, 2808, 1723, 1156, 820, 770 and 736 cm⁻¹±4 cm⁻¹, and with the lack of diffraction peaks in its powder X-ray diffractogram.

19. The compound or polymorph form of claim 17 characterized by infrared spectrum substantially depicted as in FIG. 5 and further characterized by powder X-ray diffraction diagram substantially depicted as in FIG. 10.

20. Any discretionary mixtures of Ziprasidone-hydrobromide Form I, II, III, IV and V as defined in claim 1.

21. Pharmaceutical formulation with neuroleptic activity comprising any one or any mixture of Ziprasidone-hydrobromide Form I, II, III, IV and V as defined in claim 1 for the treatment of neuroleptic diseases and a pharmaceutically acceptable carrier.

22. A process for producing a compound or polymorph form as defined in claim 1, which comprises the step or steps of:
(1) dissolving ziprasidone base in aqueous or anhydrous organic acid, in formic acid, and;
(2) reacting the solution with aqueous or anhydrous hydrogen bromide solution, and;
(3) crystallizing the Ziprasidone-hydrobromide Form I product from an aqueous solution; or
(1) reacting a formic acidic or acetic acidic solution of ziprasidone base with a hydrogen bromide solution, and;
(2) allowing the ziprasidone-hydrobromide monohydrate product to crystallize from aqueous solution; or
(1) reacting an aqueous methanolic or aqueous ethanolic suspension of ziprasidone base with hydrogen bromide solution and;
(2) allowing the ziprasidone-hydrobromide monohydrate product to crystallize with cooling; or
(1) reacting an anhydrous formic acidic solution of ziprasidone base with anhydrous hydrogen bromide solution and;
(2) allowing the ziprasidone-hydrobromide anhydrate product to crystallize with cooling; or
(1) reacting for 8-20 hour an anhydrous formic acidic solution of ziprasidone base with anhydrous hydrogen bromide solution and;
(2) crystallizing the ziprasidone-hydrobromide anhydrate product with cooling; or
heating Ziprasidone-hydrobromide Form IV, to produce ziprasidone-hydrobromide anhydrate; or
adding an anhydrous formic acidic solution of Ziprasidone-hydrobromide Form I into water at a temperature of 5-10° C., in very short time, to produce ziprasidone-hydrobromide hemihydrate; or
reacting a formic acidic solution of ziprasidone base with aqueous hydrogen bromide solution, containing organic solvents, preferably methyl isobutyl ketone, ethyl acetate, tetrahydrofuran in short time, to produce ziprasidone-sesquihydrobromide hemiformiate; or
adding hydrogen bromide solution in a mixture of glacial acetic acid and methyl isobutyl ketone into a formic acidic solution of ziprasidone base in very short time, to produce amorphous ziprasidone-hydrobromide; or
(1) dissolving ziprasidone base in an anhydrous solvent;
(2) reacting the solution with anhydrous hydrogen bromide solution, and;
(3) allowing the Ziprasidone-hydrobromide Form II product to crystallize from an anhydrous solution; or
(1) suspending ziprasidone base in an anhydrous solvent;
(2) reacting the solution with aqueous hydrogen bromide solution, and;
(3) crystallizing the Ziprasidone-hydrobromide Form II product after removing the water with boiling; or
heating Ziprasidone-hydrobromide Form IV to produce Ziprasidone-hydrobromide Form II; or
(1) dissolving ziprasidone base in formic acid;
(2) reacting the solution with glacial acetic acidic hydrogen bromide solution containing anhydrous organic acid, and;
(3) allowing the Ziprasidone-hydrobromide Form IV product to crystallize from the reaction mixture.

23. The process of claim 22 for producing ziprasidone-hydrobromide monohydrate which comprises the steps of:
(1) reacting a formic acidic or acetic acidic solution of ziprasidone base with a hydrogen bromide solution, and;
(2) allowing the product to crystallize from aqueous solution.

24. The process of claim 22 for producing ziprasidone-hydrobromide monohydrate which comprises the steps of:
(1) reacting an aqueous methanolic or aqueous ethanolic suspension of ziprasidone base with hydrogen bromide solution, for 0.5-3 hour at the boiling temperature and;
(2) allowing the product to crystallize with cooling.

25. The process of claim 22 for producing ziprasidone-hydrobromide anhydrate which comprises the steps of:
(1) reacting an anhydrous formic acidic solution of ziprasidone base with anhydrous hydrogen bromide solution, preferably with glacial acetic acidic hydrogen bromide solution at room temperature and;
(2) allowing the product to crystallize with cooling.

26. The process of claim 22 for producing ziprasidone-hydrobromide anhydrate which comprises the steps of:
(1) reacting for 8-20 hour an anhydrous formic acidic solution of ziprasidone base with anhydrous hydrogen bromide solution, with glacial acetic acidic hydrogen bromide solution at the boiling temperature and;
(2) crystallizing the product with cooling.

27. The process of claim 22 for producing ziprasidone-hydrobromide anhydrate comprising the step of heating Ziprasidone-hydrobromide Form IV, at 180-200° C.

28. The process of claim 22 for producing ziprasidone-hydrobromide hemihydrate which comprises the step of adding an anhydrous formic acidic solution of Ziprasidone-hydrobromide Form I into water at a temperature of 5-10° C., in 1 min.

29. The process of claim 22 for producing ziprasidone-sesquihydrobromide hemiformiate which comprises the step of reacting a formic acidic solution of ziprasidone base with aqueous hydrogen bromide solution, containing organic solvents, preferably methyl isobutyl ketone, ethyl acetate, tetrahydrofuran in 10-20 min.

30. The process of claim 22 for producing amorphous ziprasidone-hydrobromide which comprises the step of adding hydrogen bromide solution in a mixture of glacial acetic acid and methyl isobutyl ketone into a formic acidic solution of ziprasidone base in 1 min at a temperature of 65-70° C.

31. The process of claim 22 for producing Ziprasidone-hydrobromide Form II which comprises the steps of:
(1) dissolving ziprasidone base in an anhydrous solvent;
(2) reacting the solution with anhydrous hydrogen bromide solution, and;
(3) allowing the product to crystallize from an anhydrous solution.

32. The process of claim 22 for producing Ziprasidone-hydrobromide Form II which comprises the steps of:
(1) suspending ziprasidone base in an anhydrous solvent;
(2) reacting the solution with aqueous hydrogen bromide solution, and;
(3) crystallizing the product after removing the water with boiling.

33. The process of claim 22 for producing Ziprasidone-hydrobromide Form II which comprises heating Ziprasidone-hydrobromide Form IV.

34. The process of claim 22 for producing Ziprasidone-hydrobromide Form IV which comprises the steps of
(1) dissolving ziprasidone base in formic acid;
(2) reacting the solution with glacial acetic acidic hydrogen bromide solution containing anhydrous organic acid, and;
(3) allowing the product to crystallize from the reaction mixture.
